(19) United States
(12) Patent Application Publication
(10) Pub. No.: US 2026/0334931 A1
(43) Pub. Date: Oct. 29, 2026

(54) LAPROSCOPIC LIVER RETRACTOR AND METHOD

(71) Applicant: BOARD OF REGENTS, THE UNIVERSITY OF TEXAS SYSTEM, AUSTIN, TX (US)

(72) Inventor: Naruhiko IKOMA, Houston, TX (US)

(21) Appl. No.: 19/106,054

(22) PCT Filed: Aug. 18, 2023
(86) PCT No.: PCT/US2023/072448

§ 371 Date: Feb. 24, 2025

Related U.S. Application Data
(60) Provisional application No. 63/373,494, filed on Aug. 25, 2022.

(51) Int. Cl. A61B 17/02; A61B 90/00

(52) U.S. Cl. CPC A61B 17/0218; A61B 90/08; A61B 2017/0225; A61B 2090/0801

(57) ABSTRACT

Laparoscopic liver retractors and related methods. In one example, a laparoscopic liver retractor includes a liver support member of a stretchable and flexible support material, one or more piercing connectors associated with a perimeter of the liver support member, and two or more flexible external retraction elements associated with the perimeter of the liver support member. Also described are surgical safety fasteners that may be used, for example and not by way of limitation, in laparoscopic liver retractors and related methods.

### REFERENCE TO RELATED APPLICATION

This patent application claims the benefit of the filing date of, and priority to, U.S. Provisional Patent Application Ser. No. 63/373,494 filed Aug. 25, 2022 for a “Laproscopic Liver Retractor And Method,” the entire contents of which are hereby incorporated by this reference.

### RELATED FIELDS

Liver retractors for use in laparoscopic procedures.

### BACKGROUND

Liver retraction is a critical step to facilitate upper abdominal surgery, such as gastrectomy, anti-reflux operations (e.g. Nissen fundoplication), bariatric surgery (gastric bypass), pancreatectomy (e.g. Whipple procedure), etc. The liver is a heavy but fragile organ, and surgeons need a liver retractor that is convenient to use, and that applies gentle but strong enough retraction at a wide area of the liver. Currently-used laparoscopic liver retraction methods have significant drawbacks. For example, Nathanson retractors have a large initial cost, require significant skill and time to utilize, require a relatively large incision, limit access of other instrumentation to the operative site, and can potentially damage the liver. Modified Penrose drains have also been proposed for use as liver retractors and address some of the drawbacks of Nathanson retractors; however, the modified Penrose drain also requires a relatively high level of skill to successfully utilize. As another example, the FreeHold Trio is a commercially available liver retractor that is not in widespread use due to its use of strings in a manner that can potentially cut or damage the liver, and can also be difficult to use.

### SUMMARY

The laparoscopic liver retractors and methods introduced here provide safe and easy ways to lift and move the liver during laparoscopic upper abdominal surgery.

In one example, a laparoscopic liver retractor includes: a liver support member of a stretchable and flexible support material, the liver support member convertible between a collapsed, laparoscopic introduction configuration and an expanded, liver retraction configuration; a piercing connector associated with a perimeter of the liver support member; a first elongated, flexible external retraction element associated with the perimeter of the liver support member; and a second elongated, flexible external retraction element associated with the perimeter of the liver support member; in which the piercing connector, the first retraction element, and the second retraction element are spaced apart about the perimeter of the liver support member such that the piercing connector is located between the first and second retraction elements when the liver support member is in the expanded, liver retraction configuration; and in which the liver support member has a curved shape, with the first and second retraction elements associated with an upper half of the curved shape and the piercing connector associated with a lower half of the of the curved shape; and in which the upper half and the lower half each include an apex, with the piercing connector positioned relatively closer to the lower half apex and the first and second retraction elements positioned relatively further from the upper half apex.

The piercing connector in this example may be a surgical safety fastener including (a) an elongated pin including a piercing end, and (b) a safety cover. The safety cover may be movable between an expanded configuration in which the safety cover obstructs access to the piercing end and a retracted configuration in which the safety cover does not obstruct access to the piercing end.

The safety cover in this example may include a spring that biases the safety cover to the expanded configuration.

The safety cover in this example may further include a cover portion and a second portion in which the spring connects the cover portion to the second portion.

The cover portion in this example may have an inner wall. The inner wall may be located between the piercing end and the second portion both when the safety cover is in the expanded configuration and when the safety cover is in the retracted configuration.

The laparoscopic liver retractor in this example may use a diaphragmatic crus hook as the piercing connector.

The laparoscopic liver retractor in this example may use at least a pair of diaphragmatic crus hooks positioned adjacent each other.

The hooks used in this example may each include a piercing end and a laparoscopic manipulation portion.

In this example, the collapsed, laparoscopic introduction configuration may be a twisted configuration.

In another example, a laparoscopic liver retractor includes: a liver support member of a stretchable and flexible support material, the liver support member convertible between a collapsed, laparoscopic introduction configuration and an expanded, liver retraction configuration; a piercing connector associated with a perimeter of the liver support member; a first elongated, flexible external retraction element associated with the perimeter of the liver support member; and a second elongated, flexible external retraction element associated with the perimeter of the liver support member; in which the piercing connector, the first retraction element, and the second retraction element are spaced apart about the perimeter of the liver support member such that the piercing connector is located between the first and second retraction elements when the liver support member is in the expanded, liver retraction configuration.

The piercing connector in this example may be a diaphragmatic crus hook.

The laparoscopic liver retractor in this example may include at least a pair of diaphragmatic crus hooks positioned adjacent each other.

The hook in this example may include a piercing end and a laparoscopic manipulation portion.

The liver support member in this example may be a planar membrane.

The planar membrane in this example may be a silicone material.

The planar membrane in this example may be a disk.

The liver support member in this example may also include a reinforcement portion extending about the perimeter of the liver support member where the piercing connector and the first and second retraction elements are connected to the reinforcement portion.

The reinforcement portion in this example may include at least one of a thickened part of the flexible support member or a discrete reinforcement element.

The collapsed, laparoscopic introduction configuration of the liver support member in this example may be a twisted configuration.

In another example, a method of laparoscopic liver retraction includes: laparoscopically introducing a liver retractor into an abdominal cavity while a liver support member of the liver retractor is in a collapsed configuration; expanding the support member into an expanded, liver retraction configuration; connecting the support member to a diaphragmatic crus using a piercing connector associated with a perimeter of the liver support member; and retracting a liver in the abdominal cavity by retracting a first elongated, flexible external retraction element associated with the perimeter of the liver support member and a second elongated, flexible external retraction element associated with the perimeter of the liver support member, in which the first and second retraction elements are retracted externally of the abdominal cavity such that the support member lifts and supports the liver.

In this example, retracting the liver may include drawing the first retraction element through a first incision and drawing a second retraction element through a second incision spaced from the first incision.

In this example, connecting the support member to the diaphragmatic crus using the piercing connector may include using at least a pair of adjacently positioned diaphragmatic crus hooks to pierce the diaphragmatic crus.

In this example, the liver support member may be planar.

In this example, the perimeter of the liver support member has a curved shape, with the first and second retraction elements associated with an upper half of the curved shape and the piercing connector associated with a lower half of the of the curved shape.

In this example, the upper half and the lower half of the liver support member may each include an apex; in which the hooks are positioned relatively closer to the lower half apex and the first and second retraction elements are positioned relatively further from the upper half apex.

In this example, the support member may be positioned relative to the liver such that the first and second retraction elements extend by opposite sides of a round ligament of the liver.

In this example, retracting the liver may include retracting both the liver and the round ligament.

Also described herein is a surgical safety fastener that may be used with the laparoscopic liver retractors and methods described herein or for other uses.

### BRIEF DESCRIPTION OF FIGURES

FIG. 1 shows one example of a laparoscopic liver retractor.

FIG. 2 shows an example of a laparoscopic liver retractor in a twisted configuration.

FIG. 3 shows in cross section the liver support member of the retractor of FIG. 1.

FIG. 4 shows an example of a piercing connector.

FIG. 5 outlines an example method of laparoscopic liver retraction.

FIG. 6 shows an example of a laparoscopic liver retractor being used to lift a liver.

FIG. 7 shows an example of a surgical safety fastener, shown in an expanded configuration.

FIG. 8 shows the surgical safety fastener of FIG. 7 in a retracted configuration.

FIG. 9 shows another example of a surgical safety fastener.

FIG. 10 shows another example of a surgical safety fastener.

### DETAILED DESCRIPTION

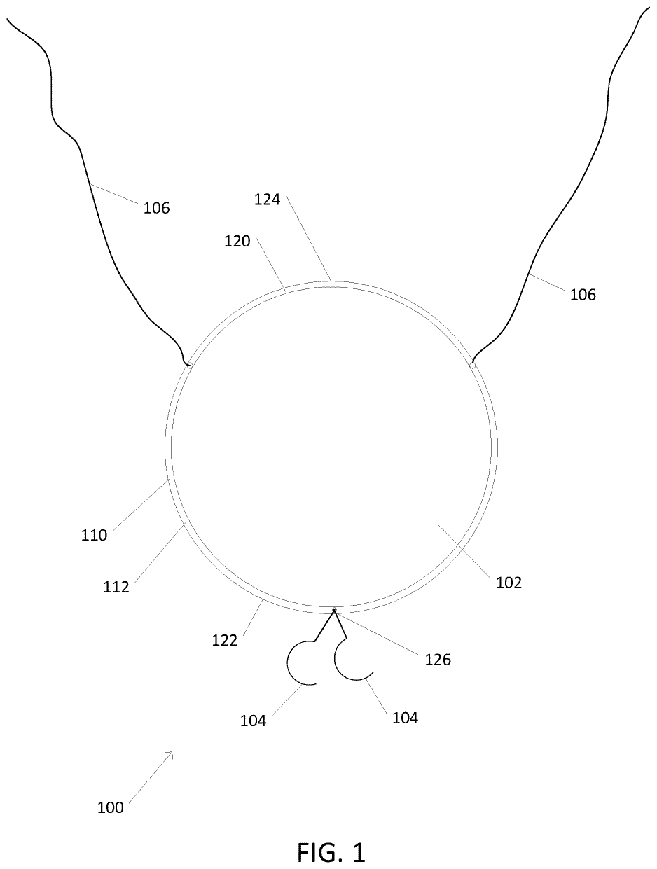

FIG. 1 shows an example of a laparoscopic liver retractor 100. The retractor may be used to facilitate liver retraction in a variety of upper abdominal surgical procedures, including, without limitation, gastrectomy (cancer, ulcer), bariatric bypass surgery (e.g. R-en-Y gastric bypass), reflux operations (e.g. Nissen fundoplication), pancreatectomy (e.g. Whipple procedure), and splenectomy. The laparoscopic liver retractor 100 provides a safe and easy way to lift and move the liver during these and other upper abdominal surgical procedures.

In the example of FIG. 1, the laparoscopic liver retractor 100 includes a liver support member 102, piercing connectors 104, and retraction elements 106.

The liver support member 102 may be formed from a stretchable and flexible support material. The liver support member 102 may be of a material that can support, lift, and move the liver with little to no damage during the surgical procedure. In one implementation, the liver support member 102 may be a disk-shaped, planar member formed of a silicone material. In other implementations, other shapes, configurations, and materials may be used. The material is soft enough that it will not damage the liver but strong enough that allows it to lift a heavy liver away from organs beneath or adjacent. In some implementations, the liver support member may be disk-shaped (circle, oval, triangular, or heart-shaped) and is flexible.

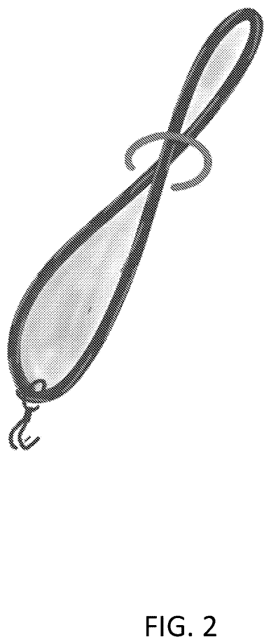

The liver support member 102 in this example is convertible between a collapsed, laparoscopic introduction configuration shown in FIG. 2 and an expanded, liver retraction configuration shown in FIG. 1. When in the expanded configuration of FIG. 1, the liver support member 102 may have a diameter or other maximum dimension in the range of 5 cm to 20 cm. The support member 102 is generally sized so that it can cover at least a portion of the liver during surgery and lift the liver upon application of force to the retraction elements 106. When in the laparoscopic introduction configuration of FIG. 2, the liver support member 102 may be twisted or otherwise reduced in size such that it can pass through a cannulated laparoscopic instrument (which may have, for example, an internal diameter in the range of 5-19 mm or in the range of 10-14 mm). An internal diameter of a cannulated laparoscopic instrument is schematically indicated at reference 108 in FIG. 2.

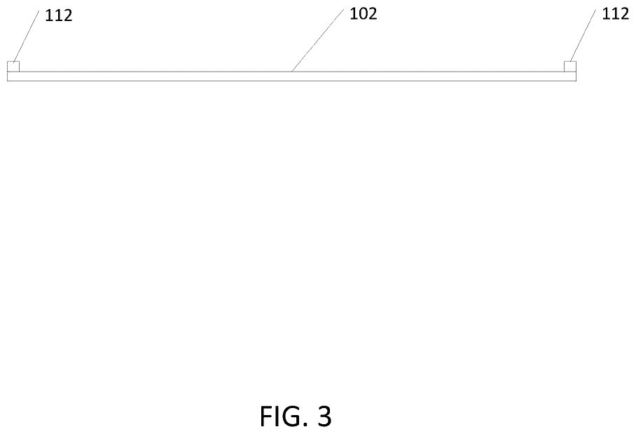

As shown in FIG. 1, the liver support member 102 includes an outer perimeter 110 surrounding a liver supporting area of the member 102. In this example, the piercing connectors 104 and the retraction elements 106 are associated with the perimeter 110 of the liver support member 102. In this example, the perimeter 110 includes a reinforcement portion 112 extending about the liver support member 102. The reinforcement portion 112 may be thicker in cross section than the rest of the liver support member 102 and is where the piercing connectors 104 and retraction elements 106 are connected. FIG. 3 shows the liver support member 102 and the thickened reinforcement portion 112 in cross section. In other implementations, the support member's perimeter may be reinforced in other ways, such as by a separate element embedded in or otherwise associated with the perimeter of the support member.

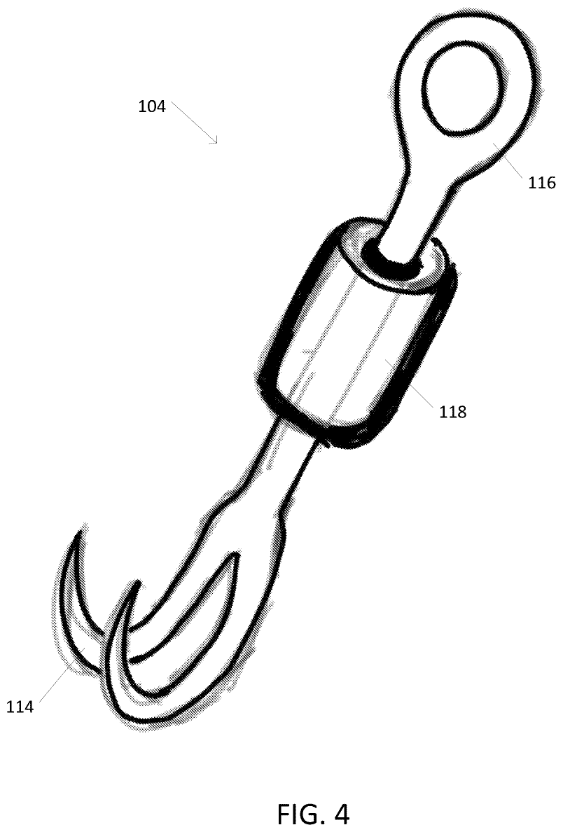

FIG. 4 shows a piercing connector 104 in more detail. In this example, the piercing connector 104 is a diaphragmatic crus hook. The piercing connector 104 is shaped, configured, and positioned relative to the other components of the retractor 100 so that the piercing connector 104 can pierce and engage the diaphragmatic crus during use. The diaphragmatic crus is a muscular and stronger area that can act as a support for the disk in use. The piercing connector 104 includes a piercing end 114 for piercing the diaphragmatic crus, a connector end 116 where it is connected to the liver support member 102, and a laparoscopic manipulation portion 118 between the piercing end 114 and the connector end 116. The laparoscopic manipulation portion 118 is configured to be engaged by a laparoscopic instrument (e.g. laparoscopic grasper/forceps).

In the example of FIG. 4, the connector end 116 includes an opening through which the liver support member 102 will be connected. The manipulation portion 118 in this example is a sleeve (e.g. of rubber or plastic) to facilitate manipulation by laparoscopic instruments. In other implementations, the manipulation portion may have a different surface texture to increase friction, or have elevated surfaces and/or protuberances shaped to correspond to a laparoscopic grasping instrument. The piercing end 114 in the example of FIG. 4 includes metallic double hooks designed to penetrate tissue such as the diaphragmatic crus. In other implementations, other numbers of hooks may be utilized. The piercing connector 104 may have a size in the range of and including 1-5 cm, with piercing end hooks having a 3-10 mm diameter.

In the example shown in FIG. 1, the retractor 100 includes a pair of piercing connectors 104 positioned adjacent each other. In this context, “adjacent” means that the pair of piercing connectors 104 are connected to the liver support member 102 with a sufficiently close spacing so that both piercing connectors 104 can engage the diaphragmatic crus. In some implementations, the piercing connectors 104 have a spacing between 0 mm and 20 mm at their connection to the liver support member 102. While FIG. 1 shows the retractor 100 with two piercing connectors 104, in other implementations other numbers of connectors may be used. In some implementations, the use of multiple piercing connectors 104 may reduce and spread out the amount of force any single connector exerts on the anatomy, lowering the risk of tearing or other undesirable levels of damage to the anatomy.

In the example shown in the figures, the retractor 100 includes a pair of retraction elements 106 connected to the support member 102. Elements 106 may be elongated and flexible, such as suture or string. Elements 106 may be sufficiently elongated so that they can extend from the support member 102 through the abdominal wall and external of the patient's anatomy so that they can be manipulated external of the anatomy to retract the liver. For example, a suture passer can be inserted through a separate incision and the surgeon can pull the retraction elements 106 though the abdominal wall to be externalized, which will be clamped at the skin level using regular surgical clamps. For instance, a hemostat or clamp can be used externally to secure the retraction elements in place once the proper amount of tension and liver height/retraction is reached.

In one implementation, the retraction elements 106 may be at least 5 cm in length.

As shown in FIG. 1, the piercing connectors 104 and the retraction elements 106 are spaced apart about the perimeter of the liver support member 102 such that the piercing connectors 104 are located between the retraction elements 106 when the liver support member 102 is in the expanded, liver retraction configuration. In this example, the perimeter 110 of the liver support member 102 has a curved shape, with the retraction elements 106 associated with an upper half 120 of the curved shape and the piercing connectors 104 associated with a lower half 122 of the of the curved shape. The upper 120 and lower 122 halves each have an apex 124, 126, with the piercing connectors 104 being positioned relatively closer to the lower half apex 126 and the retraction elements 106 positioned relatively further from the upper half apex 124. The relative positioning of the piercing connectors 104 and the retraction elements 106 facilitates optimal positioning and use of the laparoscopic liver retractor 100.

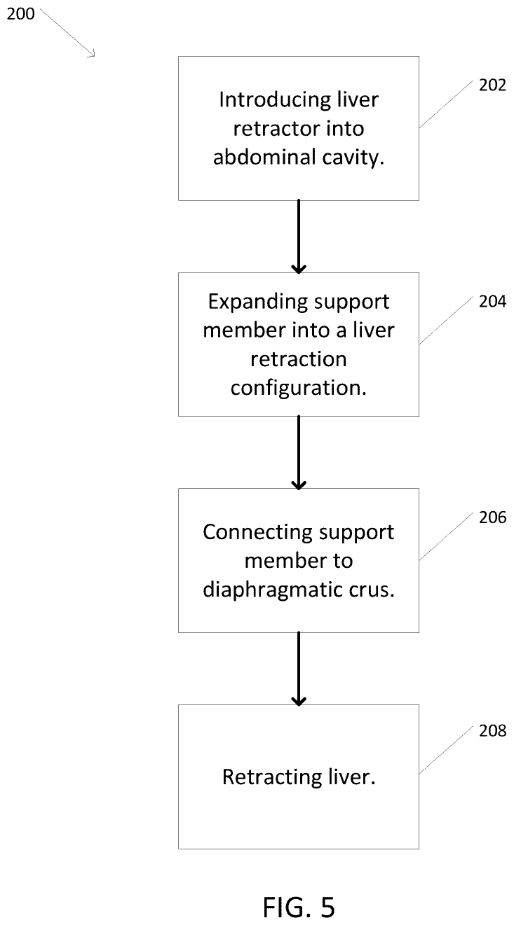

FIG. 5 outlines an example of a method of laparoscopic liver retraction 200. The method 200 may use the laparoscopic liver retractor 100 of FIGS. 1-4 or may use a differently configured retractor. The method 200 begins at step 202, which is laparoscopically introducing a liver retractor into an abdominal cavity while a liver support member of the liver retractor is in a collapsed configuration. Next, at step 204, the support member is expanded into an expanded liver retraction configuration. Next, at step 206, the support member is connected to a diaphragmatic crus using a piercing connector associated with a perimeter of the liver support member. Next, at step 208, the liver is retracted in the abdominal cavity by retracting elongated, flexible external retraction elements associated with the perimeter of the liver support member. The retraction elements are retracted externally of the abdominal cavity such that the support member lifts and supports the liver.

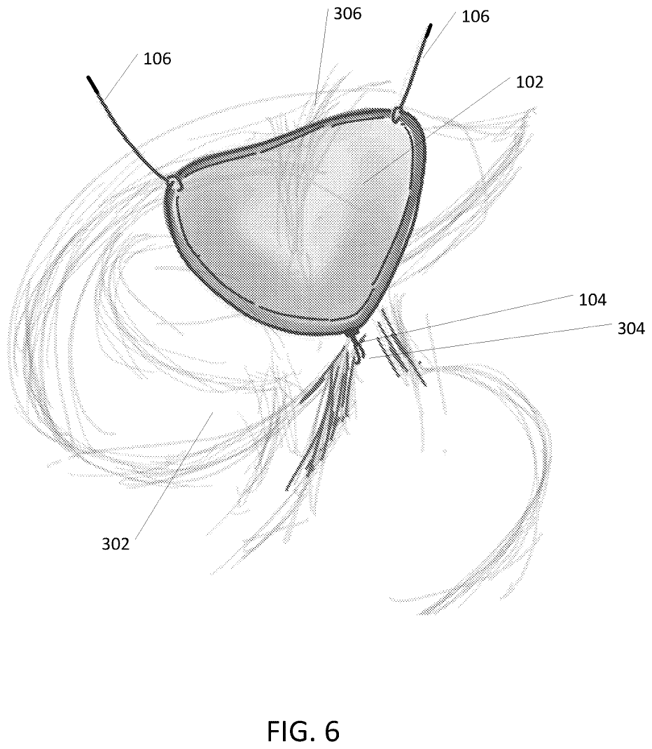

FIG. 6 shows the laparoscopic liver retractor 100 of FIGS. 1-4 being used to retract a liver 302 in accordance with the method 200 of FIG. 5. In the example of FIG. 6, piercing connectors 104 pierce and engage the diaphragmatic crus at 204 with retraction elements 106 extending out of the abdominal cavity. As can also be seen in FIG. 6, the laparoscopic liver retractor 100 is positioned so that retraction elements 106 extend by opposite sides of the round ligament of the liver 306 such that both the round ligament of the liver 306 and the liver 302 can be retracted at the same time. As can be seen in FIG. 6, although the support member of the retractor is originally circular in shape, pressure from the piercing connectors at the bottom and the retraction elements at the top can cause the support member to take on somewhat of a triangular shape in use.

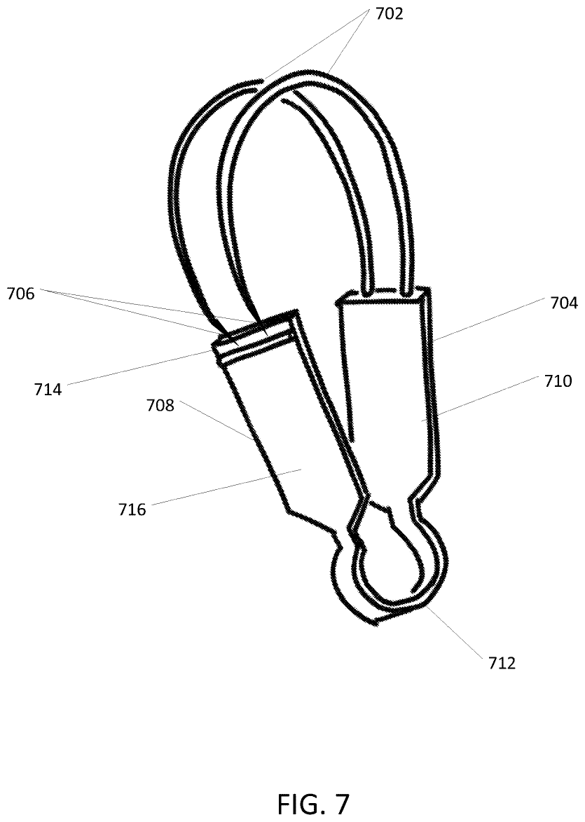

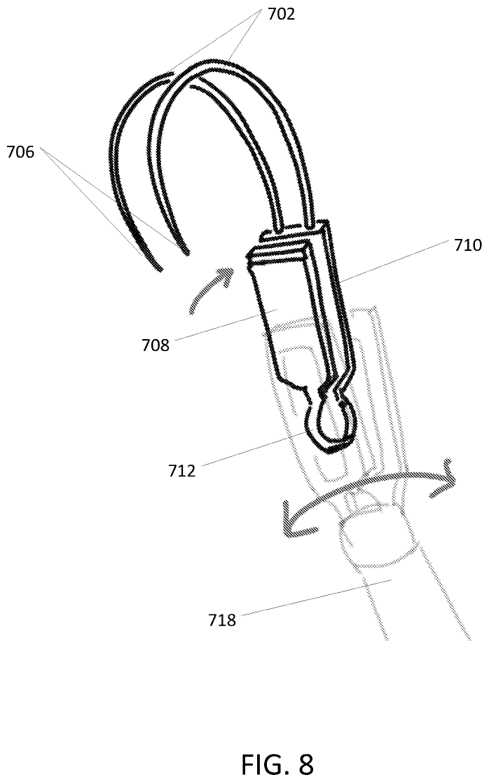

FIGS. 7 and 8 show a surgical safety fastener that may be used as a piercing connector instead of the piercing connection 104 shown in FIG. 4. The surgical safety fastener of FIGS. 7 and 8 may be used with laparoscopic liver retractors such as the examples described above. The surgical safety fastener of FIGS. 7 and 8 may also be used for other uses.

In the example of FIG. 7, the surgical safety fastener includes two elongated pins 702 and a safety cover 704. Each of the elongated pins 702 includes a piercing end 706. The safety cover 704 is movable between an expanded configuration (shown in FIG. 7) in which the safety cover 704 obstructs access to the piercing ends 706 and a retracted configuration (shown in FIG. 8) in which the safety cover 704 does not obstruct access to the piercing end.

The safety cover includes a cover portion 708, a second portion 710, and a spring 712. The spring 712 connects the cover portion 708 to the second portion 710 and biases the safety cover 704 to its expanded configuration. The cover portion 708 includes an inner wall 714. The inner wall 714 is located between the piercing end 706 of the elongated pins 702 and the second portion 710 of the safety cover 704 both when the safety cover 704 is in the expanded configuration (as shown in FIG. 7) and when the safety cover 704 is in the retracted configuration (as shown in FIG. 8).

In the example of FIGS. 7 and 8, the safety cover 704 is squeezable to move the cover portion 708 towards the second portion 710 and away from the piercing ends 706 of the elongated pins 702, thereby moving the safety cover 704 from the expanded configuration shown in FIG. 7 to the retracted configuration shown in FIG. 8. When in the retracted configuration, the piercing ends 706 are exposed (e.g. such that they can pierce the diaphragmatic crus or other anatomy to which the surgical safety fastener is to be secured). When the safety cover 704 is released, the safety cover 704 will return to the expanded configuration 704 such that the piercing ends 706 are obstructed and prevented from unintentionally piercing the patient's anatomy or anything else.

The safety cover 704 includes grasping surfaces 716 facilitating manipulation of the fastener using grasping instrumentation (such as the robotic fenestrated grasper 718 shown in FIG. 8). In the particular example shown in FIGS. 7 and 8, the grasping surfaces 716 are planar surfaces although other configurations may be possible to optimize interaction with a grasping instrument. The shape and size of the fastener may otherwise be configured for use with common robotic grasping instruments.

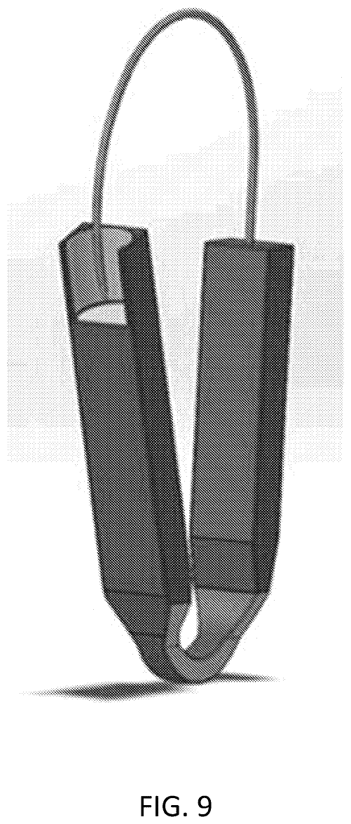

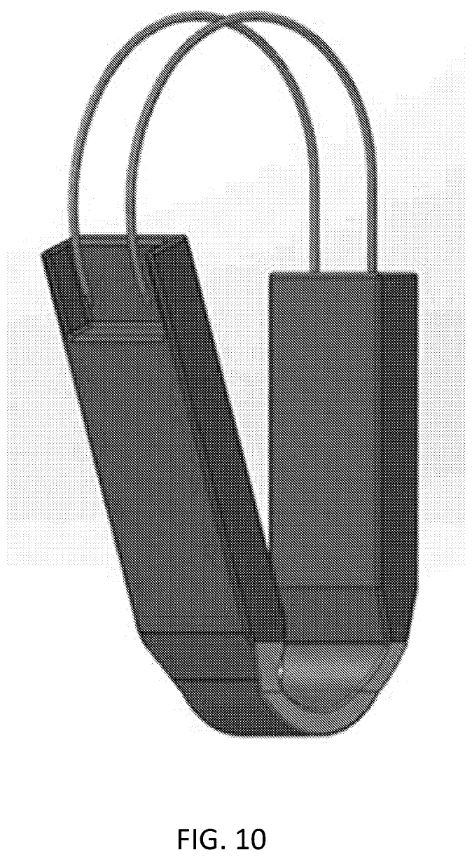

In the example shown in FIGS. 7 and 8, the inner wall 714 is part of a ledge formed in the cover portion 708 that is configured to obstruct access to the piercing ends 706 of the elongated pins 702 when the safety cover 704 is in the expanded configuration. In the examples shown in FIGS. 9 and 10, the inner wall 714 is part of a cavity formed in the cover portion 708 that is configured to obstruct access to the piercing ends 706 of the elongated pin(s) 702 when the safety cover 704 is in the expanded configuration. In all of the examples shown in FIGS. 7-10, the inner wall 714 interacts with the piercing end(s) 706 to both obstruct access to them and to also limit how far the cover portion 708 can move away from the second portion 710 in response to the biasing force applied by spring 712.

In some implementations, the cover portion 708, second portion 710, and spring 712 may all be part of a unitarily formed body. In other implementations, the cover portion 708, second portion 710, and spring 712 may be discrete components joined together with one another.

As shown in the examples of FIGS. 7-10, the pin(s) 702 extend from the second portion and curve back towards the cover portion 708. As shown in FIGS. 7-10, the safety fastener may include one or multiple pins 702.

The laparoscopic liver retractors and methods described above and the surgical safety fasteners described above are provided as examples. Additions, deletions, substitutions, modifications, and other changes may be made without departing from the scope or spirit of the present inventions.

1. A laparoscopic liver retractor, comprising:
(a) a liver support member comprising a stretchable and flexible support material, the liver support member convertible between a collapsed, laparoscopic introduction configuration and an expanded, liver retraction configuration, the liver support member comprising a liver supporting area surrounded by an outer perimeter;
(b) a piercing connector associated with the outer perimeter of the liver support member;
(c) a first elongated, flexible external retraction element associated with the outer perimeter of the liver support member; and
(d) a second elongated, flexible external retraction element associated with the outer perimeter of the liver support member;
wherein the piercing connector, the first retraction element, and the second retraction element are spaced apart about the outer perimeter of the liver support member such that the piercing connector is located between the first and second retraction elements when the liver support member is in the expanded, liver retraction configuration;
wherein liver support member comprises a curved shape, with the first and second retraction elements associated with an upper half of the curved shape and the piercing connector associated with a lower half of the of the curved shape; and
wherein the upper half and the lower half each comprise an apex, with the piercing connector positioned relatively closer to the lower half apex and the first and second retraction elements positioned relatively further from the upper half apex.

2. The laparoscopic liver retractor of claim 1, wherein the piercing connector comprises a surgical safety fastener comprising:
(a) an elongated pin comprising a piercing end; and
(b) a safety cover movable between an expanded configuration in which the safety cover obstructs access to the piercing end and a retracted configuration in which the safety cover does not obstruct access to the piercing end.

3. The laparoscopic liver retractor of claim 2, wherein the safety cover further comprises a spring that biases the safety cover to the expanded configuration.

4. The laparoscopic liver retractor of claim 3, wherein the safety cover further comprises a cover portion and a second portion, wherein the spring connects the cover portion to the second portion.

5. The laparoscopic liver retractor of claim 4, wherein the cover portion comprises an inner wall, wherein the inner wall is located between the piercing end and the second portion both when the safety cover is in the expanded configuration and when the safety cover is in the retracted configuration.

6. The laparoscopic liver retractor of claim 1, wherein the piercing connector comprises a diaphragmatic crus hook.

7. The laparoscopic liver retractor of claim 1, wherein the collapsed, laparoscopic introduction configuration of the liver support member comprises a twisted configuration.

8. A laparoscopic liver retractor, comprising:
(a) a liver support member comprising a stretchable and flexible support material, the liver support member convertible between a collapsed, laparoscopic introduction configuration and an expanded, liver retraction configuration, the liver support member comprising a liver supporting area surrounded by an outer perimeter;
(b) a piercing connector associated with the outer perimeter of the liver support member;
(c) a first elongated, flexible external retraction element associated with the outer perimeter of the liver support member; and
(d) a second elongated, flexible external retraction element associated with the outer perimeter of the liver support member, the piercing connector;
wherein the first retraction element, and the second retraction element are spaced apart about the outer perimeter of the liver support member such that the piercing connector is located between the first and second retraction elements when the liver support member is in the expanded, liver retraction configuration.

9. The laparoscopic liver retractor of claim 8, wherein the piercing connector comprises a diaphragmatic crus hook.

10. The laparoscopic liver retractor of claim 9, wherein the diaphragmatic crus hook comprises at least a pair of diaphragmatic crus hooks positioned adjacent each other.

11. The laparoscopic liver retractor of claim 10, wherein the hooks each comprise a piercing end and a laparoscopic manipulation portion.

12. The laparoscopic liver retractor of claim 10, wherein the liver support member comprises a planar membrane.

13. The laparoscopic liver retractor of claim 12, wherein the planar membrane comprises a silicone material.

14. The laparoscopic liver retractor of claim 12, wherein the planar membrane comprises a disk.

15. The laparoscopic liver retractor of claim 10, wherein the liver support member further comprises a reinforcement portion extending about the outer perimeter of the liver support member, wherein the piercing connector and the first and second retraction elements are connected to reinforcement portion.

16. The laparoscopic liver retractor of claim 15, wherein the reinforcement portion comprises at least one of a thickened part of the flexible support member or a discrete reinforcement element.

17. The laparoscopic liver retractor of claim 10, wherein the collapsed, laparoscopic introduction configuration of the liver support member comprises a twisted configuration.

18. A method of laparoscopic liver retraction, comprising:
(a) laparoscopically introducing a liver retractor into an abdominal cavity while a liver support member of the liver retractor is in a collapsed configuration;
(b) expanding the support member into an expanded, liver retraction configuration;
(c) connecting the support member to a diaphragmatic crus using a piercing connector associated with an outer perimeter of the liver support member; and
(d) retracting a liver in the abdominal cavity by retracting a first elongated, flexible external retraction element associated with the outer perimeter of the liver support member and a second elongated, flexible external retraction element associated with the outer perimeter of the liver support member, wherein the first and second retraction elements are retracted externally of the abdominal cavity such that the support member lifts and supports the liver.

19. The method of claim 18, wherein retracting the liver comprises drawing the first retraction element through a first incision and drawing a second retraction element through a second incision spaced from the first incision.

20. The method of claim 19, wherein connecting the support member to the diaphragmatic crus using the piercing connector comprises using at least a pair of adjacently positioned diaphragmatic crus hooks to pierce the diaphragmatic crus.

21. The method of claim 20, wherein the liver support member is planar.

22. The method of claim 21, wherein the perimeter of the liver support member comprises a curved shape, with the first and second retraction elements associated with an upper half of the curved shape and the piercing connector associated with a lower half of the of the curved shape.

23. The method of claim 22, wherein the upper half and the lower half each comprise an apex; wherein the hooks are positioned relatively closer to the lower half apex and the first and second retraction elements are positioned relatively further from the upper half apex.

24. The method of claim 23, wherein the support member is positioned relative to the liver such that the first and second retraction elements extend by opposite sides of a round ligament of the liver.

25. The method of claim 24, wherein retracting the liver comprises retracting the liver and retracting the round ligament.

26. A surgical safety fastener comprising:
(a) an elongated pin comprising a piercing end; and
(b) a safety cover movable between an expanded configuration in which the safety cover obstructs access to the piercing end and a retracted configuration in which the safety cover does not obstruct access to the piercing end.

27. The surgical safety fastener of claim 26, wherein the safety cover further comprises a spring that biases the safety cover to the expanded configuration.

28. The surgical safety fastener of claim 27, wherein the safety cover further comprises a cover portion and a second portion, wherein the spring connects the cover portion to the second portion.

29. The surgical safety fastener of claim 28, wherein the cover portion comprises an inner wall, wherein the inner wall is located between the piercing end and the second portion both when the safety cover is in the expanded configuration and when the safety cover is in the retracted configuration.

30. The surgical safety fastener of claim 29, the cover portion further comprising a cavity for receiving the piercing end when the safety cover is in the expanded configuration, wherein the inner wall is located in the cavity.

31. The surgical safety fastener of claim 28, wherein the pin extends from the second portion.

32. The surgical safety fastener of claim 31, wherein the pin curves back towards the cover portion.

33. The surgical safety fastener of claim 28, wherein the safety cover is squeezable to move the cover portion towards the second portion and away from the piercing end of the elongated pin, thereby moving the safety cover from the expanded configuration to the retracted configuration.

34. The surgical safety fastener of claim 28, wherein the cover portion comprises a grasping surface, wherein the second portion comprises another grasping surface.

35. The surgical safety fastener of claim 34, wherein the grasping surfaces of the cover and second portions are planar grasping surfaces.
